(19) United States
(12) Patent Application Publication
(10) Pub. No.: US 2016/0117062 A1
(43) Pub. Date: Apr. 28, 2016

(54) Visualizations of Medical Inventory

(71) Applicant: Universal Research Solutions, LLC, Columbia, MO (US)

(72) Inventors: Ali Adel Hussam, Columbia, MO (US); Nathan Bleigh, Kansas City, MO (US)

(21) Appl. No.: 14/919,978

(22) Filed: Oct. 22, 2015

Related U.S. Application Data
(60) Provisional application No. 62/067,794, filed on Oct. 23, 2014.

(51) Int. Cl. G06F 3/0482; A61B 19/00; G06F 3/0484

(52) U.S. Cl. CPC G06F 3/0482; G06F 3/04842; A61B 19/56

(57) ABSTRACT

A computer-implemented method comprising: determining types of items that are required to perform a particular type of surgery; generating data for a graphical user interface that when rendered on a display device, displays: a surgical tray visualization of a surgical tray, with the surgical tray visualization comprising: one or more required material visualizations of the one or more types of items that are required to perform a particular type of surgery, with a required material visualization specifying how many of a particular type of material or instrument is required for the surgery and further specifying how many of the particular type of material or instrument has been virtually played on the surgical tray; for a type of item required, item placement information, selection of which causes the surgical tray representation to be updated to account for the selected item and to virtually place the selected item on the surgical tray.

### CLAIM OF PRIORITY

This application claims priority under 35 U.S.C. §119(e) to provisional U.S. Patent Application No. 62/067,794, filed on Oct. 23, 2014, the entire contents of which are hereby incorporated by reference.

### BACKGROUND

Radio-frequency identification (RFID) is the wireless use of electromagnetic fields to transfer data, for the purposes of automatically identifying and tracking tags attached to objects.

### SUMMARY

In an example, a computer-implemented method comprises determining, based on contents of a request, one or more types of items that are required to perform a particular type of surgery; generating, by one or more computer systems, data for a graphical user interface that when rendered on a display device, displays: a surgical tray visualization of a surgical tray, with the surgical tray visualization comprising: one or more required material visualizations of the one or more types of items that are required to perform a particular type of surgery, with a required material visualization specifying how many of a particular type of material or instrument is required for the surgery and further specifying how many of the particular type of material or instrument has been virtually played on the surgical tray; and for a type of item required, item placement information, selection of which causes the surgical tray representation to be updated to account for the selected item and to virtually place the selected item on the surgical tray. A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

### BRIEF DESCRIPTION OF THE FIGURES

FIG. 1 is an example of a graphical user interface for displaying visualizations of medical inventory.

FIG. 2 is a block diagram of components of a system for displaying visualizations of medical inventory.

### DETAILED DESCRIPTION

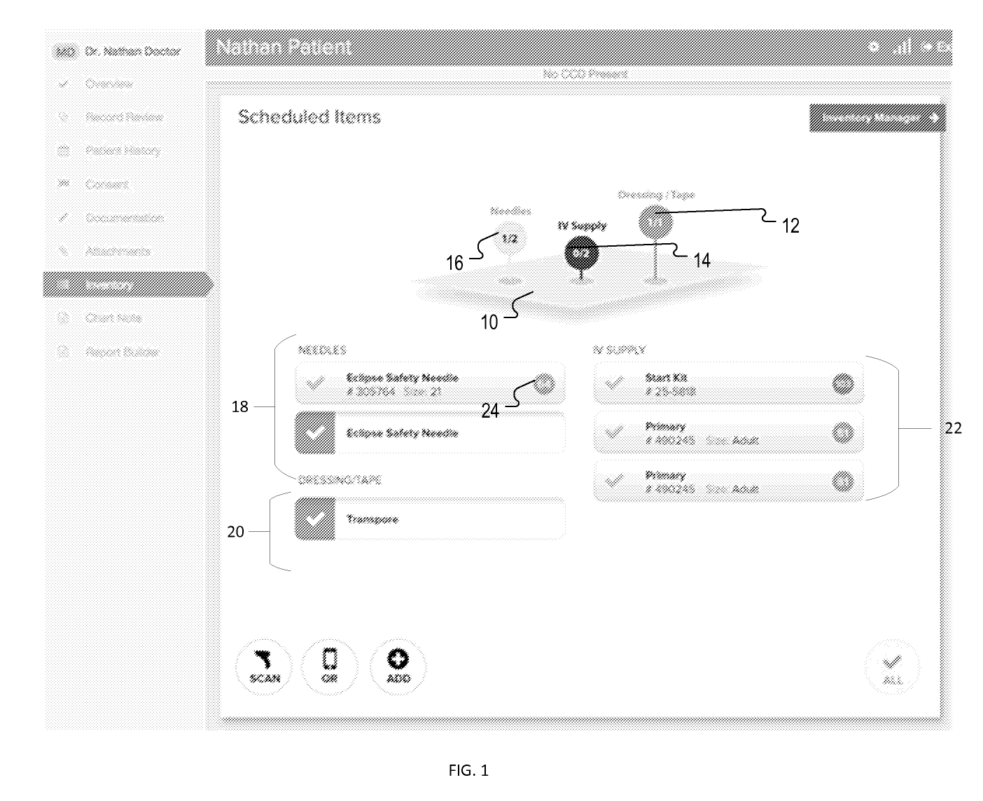

Referring to FIG. 1, a graphical user interface displays visualization 10 a surgical tray and visualizations 12, 14, 16 of items that are needed to perform a particular type of surgery. For example, visualization 12 specifies that dressing/tape are needed. Visualization 12 also includes the numbers “1/1” to specify that 1 type of dressing/tape is needed (the second “1”) and to specify that the user has selected to place the one type of dressing/tape onto the surgery tray (the first one). In this example, visualization 12 is color coded “green” to specify that one type of dressing tape is needed and the needed type of dressing tape is placed on the surgical tray.

Visualization 14 specifies that IV supplies are needed. In particular, visualization 14 specifies that 2 IV supplies are needed and that 0 IV supplies have been placed on the tray. Accordingly, visualization 14 is color coded black to specify that additional action is needed. Visualization 16 specifies that two types of needles are needed and that 1 type of needle has been placed on the tray. Visualization 16 is color coded yellow to specify that additional action is needed, e.g., by placing more needle items on the tray. Visualization 16 is color coded yellow, and not black, to specify a less severe required action.

The graphical user interface also includes portions 18, 20, 22. Portion 18 specifies the types of needles to be selected to be added to the tray. Upon selection of one of the types of needles, visualization 16 is updated. Inventory information 24 specifies the quantity of that particular type of needle that is in inventory. If the amount of needle inventory drops low, inventor information 24 will turn red or another color to alert the user to the low level of inventory. Portion 20 specifies the type of dressing tape that can be selected to be added to the tray. Portion 22 specifies the types of IV supplies to be added to the tray. In this example, visualization 14 specifies that it is recommended to have two IV supplies. A user has three different options to selected from, as shown in portion 22.

After the user puts on the virtual surgical tray all of the items that they want to use, a client device that displays the graphical user interface transmits to an inventory system a request that includes information indicative of the requested items. The inventory system then alerts an inventory control manager or other user of the requested items, to enable the user to procure them for surgery.

The inventory room includes a scanner for scanning RFID tags (or other identifying information) on an item of inventory, e.g, when the item of inventory is removed from the inventory room—to enable the inventory system to keep an accurate tally of much stock of each item is in the inventory room. That is, when a user retrieves an item of inventory, the user scans the RFID tag to alert an inventory room system that there is one less item. The inventory room system generates an updated metric specifying the remaining quantity for the particular item. An updated metric regarding the amount of inventory is transmitted, via the network, to the systems described herein to enable updating, in real time, of the described graphical user interfaces.

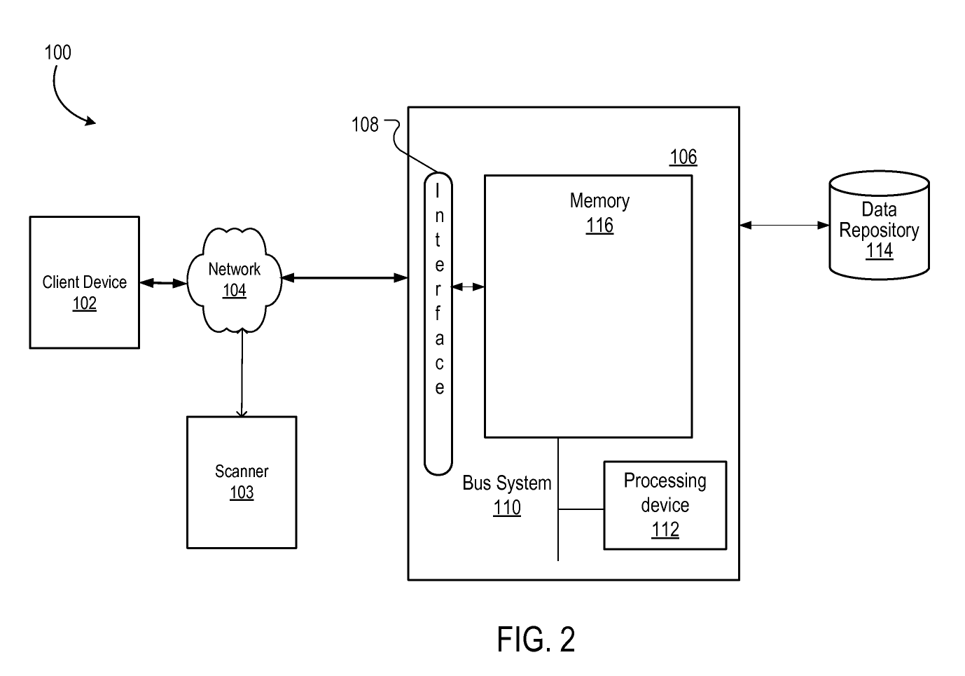

Referring to FIG. 2, networked environment 100 tracks supply of surgical items and generates graphical user interfaces, e.g., such as those shown in FIG. 1. Client device 102 can be any sort of computing device capable of taking input from a user and communicating over network 104 with system 106. In this example, system 106 tracks inventory and generates data for graphical user interfaces that display inventory information and enable users to select inventor. For example, client device 102 can be a mobile device, a desktop computer, a laptop, a cell phone, a personal digital assistant (“PDA”), a server, an embedded computing system, and so forth.

System 106 can be any of a variety of computing devices capable of receiving data, such as a server, a distributed computing system, a desktop computer, a laptop, a cell phone, a rack-mounted server, and so forth. System 106 may be a single server or a group of servers that are at a same location or at different locations.

The illustrated system 106 can receive data from client device 102 via input/output (“I/O”) interface 108. I/O interface 108 can be any type of interface capable of receiving data over a network, such as an Ethernet interface, a wireless networking interface, a fiber-optic networking interface, a modem, and so forth. System 106 also includes a processing device 112 and memory 116. A bus system 110, including, for example, a data bus and a motherboard, can be used to establish and to control data communication between the components of system 106.

The illustrated processing device 112 may include one or more microprocessors. Generally, processing device 112 may include any appropriate processor and/or logic that is capable of receiving and storing data, and of communicating over a network (not shown). Memory 116 can include a hard drive and a random access memory storage device, such as a dynamic random access memory, or other types of non-transitory machine-readable storage devices. Memory 116 stores computer programs (not shown) that are executable by processing device 112 to perform the techniques described herein. Networked environment 100 also includes scanner 103 (e.g., in an inventory room) for a user to scan items that are taken out of inventory. In this example, client device 102 is located in a surgical center. In this example, data repository 114 includes information specifying a mapping of different types of surgeries to different types of tools/materials that are required and/or necessary for the surgery. In this example, when a user inputs into the system the type of surgery they are performing, system 106 accesses the mapping to determine needed items (and types of items) and to generate the visualizations shown in FIG. 1.

The features described herein constitute an improvement to the technical field of dynamically displaying visual representations of medical inventory information. One of the problems addressed by these techniques is that of dynamically selecting medical inventory required for surgeries and being presented with real time information specifying a current inventory level, to enable medical professionals to make realistic decisions about which supplies may currently be obtained and to notify administrators regarding which supplies need to be refilled. The techniques described herein solve these challenges by dynamically tracking inventory amounts and displaying these amounts in a graphical user interface, as a user is selecting which supplies should be fulfilled and obtained for surgical use.

Embodiments can be implemented in digital electronic circuitry, or in computer hardware, firmware, software, or in combinations thereof. An apparatus can be implemented in a computer program product tangibly embodied or stored in a machine-readable storage device for execution by a programmable processor; and method actions can be performed by a programmable processor executing a program of instructions to perform functions by operating on input data and generating output. The embodiments described herein, and other embodiments of the invention, can be implemented advantageously in one or more computer programs that are executable on a programmable system including at least one programmable processor coupled to receive data and instructions from, and to transmit data and instructions to, a data storage system, at least one input device, and at least one output device. Each computer program can be implemented in a high-level procedural or object oriented programming language, or in assembly or machine language if desired; and in any case, the language can be a compiled or interpreted language.

Processors suitable for the execution of a computer program include, by way of example, both general and special purpose microprocessors, and any one or more processors of any kind of digital computer. Generally, a processor will receive instructions and data from a read-only memory or a random-access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. Computer readable media for embodying computer program instructions and data include all forms of non-volatile memory, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in special purpose logic circuitry. Any of the foregoing can be supplemented by, or incorporated in, ASICs (application-specific integrated circuits).

To provide for interaction with a user, embodiments can be implemented on a computer having a display device, e.g., a LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Embodiments can be implemented in a computing system that includes a back end component, e.g., as a data server, or that includes a middleware component, e.g., an application server, or that includes a front end component, e.g., a client computer having a graphical user interface or a Web browser through which a user can interact with an implementation of embodiments, or any combination of such back end, middleware, or front end components. The components of the system can be interconnected by any form or medium of digital data communication, e.g., a communication network. Examples of communication networks include a local area network (LAN) and a wide area network (WAN), e.g., the Internet.

The system and method or parts thereof may use the “World Wide Web” (Web or WWW), which is that collection of servers on the Internet that utilize the Hypertext Transfer Protocol (HTTP). HTTP is a known application protocol that provides users access to resources, which may be information in different formats such as text, graphics, images, sound, video, Hypertext Markup Language (HTML), as well as programs. Upon specification of a link by the user, the client computer makes a TCP/IP request to a Web server and receives information, which may be another Web page that is formatted according to HTML. Users can also access other pages on the same or other servers by following instructions on the screen, entering certain data, or clicking on selected icons. It should also be noted that any type of selection device known to those skilled in the art, such as check boxes, drop-down boxes, and the like, may be used for embodiments using web pages to allow a user to select options for a given component. Servers run on a variety of platforms, including UNIX machines, although other platforms, such as Windows 2000/2003, Windows NT, Sun, Linux, and Macintosh may also be used. Computer users can view information available on servers or networks on the Web through the use of browsing software, such as Firefox, Netscape Navigator, Microsoft Internet Explorer, or Mosaic browsers. The computing system can include clients and servers. A client and server are generally remote from each other and typically interact through a communication network. The relationship of client and server arises by virtue of computer programs running on the respective computers and having a client-server relationship to each other.

Other embodiments are within the scope and spirit of the description claims. For example, due to the nature of software, functions described above can be implemented using software, hardware, firmware, hardwiring, or combinations of any of these. Features implementing functions may also be physically located at various positions, including being distributed such that portions of functions are implemented at different physical locations. The use of the term “a” herein and throughout the application is not used in a limiting manner and therefore is not meant to exclude a multiple meaning or a “one or more” meaning for the term “a.” Additionally, to the extent priority is claimed to a provisional patent application, it should be understood that the provisional patent application is not limiting but includes examples of how the techniques described herein may be implemented.

A number of exemplary embodiments of the invention have been described. Nevertheless, it will be understood by one of ordinary skill in the art that various modifications may be made without departing from the spirit and scope of the invention.

What is claimed is:

1. A computer-implemented method comprising:
determining, based on contents of a request, one or more types of items that are required to perform a particular type of surgery;
generating, by one or more computer systems, data for a graphical user interface that when rendered on a display device, displays:
a surgical tray visualization of a surgical tray, with the surgical tray visualization comprising:
one or more required material visualizations of the one or more types of items that are required to perform a particular type of surgery, with a required material visualization specifying how many of a particular type of material or instrument is required for the surgery and further specifying how many of the particular type of material or instrument has been virtually played on the surgical tray; and
for a type of item required, item placement information, selection of which causes the surgical tray representation to be updated to account for the selected item and to virtually place the selected item on the surgical tray.
